(19) United States
(12) Patent Application Publication
(10) Pub. No.: US 2025/0090220 A1
(43) Pub. Date: Mar. 20, 2025

(54) METHODS AND SYSTEMS FOR ADVANCED HARMONIC ENERGY

(71) Applicant: Cilag GmbH International, Zug (CH)

(72) Inventors: David C. Yates, Morrow, OH (US); Amy M. Krumm, Cincinnati, OH (US); Mark A. Davison, Maineville, OH (US)

(21) Appl. No.: 18/894,131

(22) Filed: Sep. 24, 2024

Related U.S. Application Data
(63) Continuation of application No. 17/750,816, filed on May 23, 2022, now Pat. No. 12,114,914.
Application No. 17/750,816 is a continuation of application No. 16/531,591, filed on Aug. 5, 2019, now Pat. No. 11,344,362.
Application No. 16/531,591 is a continuation of application No. 15/229,562, filed on Aug. 5, 2016, now Pat. No. 10,376,305.

(51) Int. Cl. A61B 18/14; A61B 17/32; A61B 18/00; A61B 18/12

(52) U.S. Cl. CPC A61B 18/1445; A61B 17/320092; A61B 18/1206; A61B 18/1482; A61B 2017/320094; A61B 2017/320095; A61B 2018/00208; A61B 2018/0063; A61B 2018/00642; A61B 2018/00678; A61B 2018/00702; A61B 2018/00875; A61B 2018/00994

(57) ABSTRACT

Aspects of the present disclosure are presented for a medical instrument configured to adjust the power level for sealing procedures to account for changes in tissue impedance levels over time. In some aspects, a medical instrument may be configured to apply power according to a power algorithm to seal tissue by applying a gradually lower amount of power over to time as the tissue impedance level begins to rise out of the “bathtub region,” which is the time period during energy application where the tissue impedance is low enough for electrosurgical energy to be effective for sealing tissue. In some aspects, the power is then cut once the tissue impedance level exceeds the “bathtub region.” By gradually reducing the power, a balance is achieved between still applying an effective level of power for sealing and prolonging the time in which the tissue impedance remains in the “bathtub region.”

### CROSS-REFERENCE TO RELATED APPLICATIONS

This application is a continuation application claiming priority under 35 U.S.C. § 120 to U.S. patent application Ser. No. 17/750,816, entitled METHODS AND SYSTEMS FOR ADVANCED HARMONIC ENERGY, filed May 23, 2022, now U.S. Patent Application Publication No. 2022/0346863, which is a continuation application claiming priority under 35 U.S.C. § 120 to U.S. patent application Ser. No. 16/531,591, entitled METHODS AND SYSTEMS FOR ADVANCED HARMONIC ENERGY, filed Aug. 5, 2019, which issued on May 31, 2022 as U.S. Pat. No. 11,344,362, which is a continuation application claiming priority under 35 U.S.C. § 120 to U.S. patent application Ser. No. 15/229,562, entitled METHODS AND SYSTEMS FOR ADVANCED HARMONIC ENERGY, filed Aug. 5, 2016, which issued on Aug. 13, 2019 as U.S. Pat. No. 10,376,305, the entire disclosures of which are hereby incorporated by reference herein.

### TECHNICAL FIELD

The present disclosure is related generally to medical devices with various mechanisms for grasping and sealing tissue. In particular, the present disclosure is related to electrosurgical instruments configured to regulate the application of energy applied to a surgical site to prolong the sealing time duration when performing sealing procedures.

### BACKGROUND

Electrosurgical instruments are a type of surgical instrument used in many surgical operations. Electrosurgical instruments apply electrical energy to tissue in order to treat tissue. An electrosurgical instrument may comprise an instrument having a distally-mounted end effector comprising one or more electrodes. The end effector can be positioned against tissue such that electrical current is introduced into the tissue. Electrosurgical instruments can be configured for bipolar or monopolar operation. During bipolar operation, current is introduced into and returned from the tissue by active and return electrodes, respectively, of the end effector. During monopolar operation, current is introduced into the tissue by an active (or source) electrode of the end effector and returned through a return electrode (e.g., a grounding pad) separately located on a patient's body. Heat generated by the current flow through the tissue may form hemostatic seals within the tissue and/or between tissues and thus may be particularly useful for sealing blood vessels, for example. The end effector of an electrosurgical instrument sometimes also comprises a cutting member that is moveable relative to the tissue and the electrodes to transect the tissue.

Energy applied by an electrosurgical instrument can be transmitted to the instrument by a generator. The generator may form an electrosurgical signal that is applied to an electrode or electrodes of the electrosurgical instrument. The generator may be external or integral to the electrosurgical instrument. The electrosurgical signal may be in the form of radio frequency (“RF”) energy. For example, RF energy may be provided at a frequency range of between 100 kHz and 1 MHz. During operation, an electrosurgical instrument can transmit RF energy through tissue, which causes ionic agitation, or friction, in effect resistive heating, thereby increasing the temperature of the tissue. Because a sharp boundary may be created between the affected tissue and the surrounding tissue, surgeons can operate with a high level of precision and control, without sacrificing un-targeted adjacent tissue. The low operating temperatures of RF energy may be useful for removing, shrinking, or sculpting soft tissue while simultaneously sealing blood vessels. RF energy may work particularly well on connective tissue, which is primarily comprised of collagen and shrinks when contacted by heat. In some cases, the instrument may also be configured to apply ultrasonic energy to create homeostasis. The generator may be configured to transmit energy which is converted into ultrasonic vibrations at the end effector. The energy transmitted to the tissue may similarly cause resistive heating through the ultrasonic vibrations.

During the application of the energy to tissue, the impedance of the tissue indicates the condition of the tissue. After a certain amount of energy applied, the impedance of the tissue dramatically increases and reduces the effectiveness of the further energy applied in the sealing procedure. Furthermore, as the tissue impedance approaches this threshold level where further energy applied is no longer effective, certain chemical processes in the tissue occur that would be desirable to be controlled better. The period of time under which the tissue responds to the sealing energy is sometimes referred to as the “bathtub region,” based on the shape of the level of impedance over time at which the tissue effectively responds to the sealing energy. There is a need therefore to better control the rise of impedance levels in the tissue and to prolong the period under which the tissue still responds (e.g., prolong the “bathtub region”) to applied energy during sealing procedures. While several devices have been made and used, it is believed that no one prior to the inventors has made or used the device described in the appended claims.

### SUMMARY

In some aspects, a surgical system is provided.

In one aspect, the surgical system may include: an end effector comprising at least one energy delivery component configured to transmit electrosurgical energy at a number of different power levels (i.e., rates of energy delivery or levels of energy delivery) to tissue at a surgical site; and a control circuit communicatively coupled to the energy delivery component and programmed to: for a first application period, cause the energy delivery component to transmit the electrosurgical energy at a first power level or rate of energy delivery, the first application period comprising a point in time where impedance of the tissue reaches a minimum value; for a second application period after the first application period, cause the energy delivery component to transmit the electrosurgical energy at a decreasing power level or rate of energy level from the first power level until a second power level is reached, the second power level lower than the first power level and the second application period comprising a point in time where the impedance of the tissue rises above the minimum impedance value; for a third application period after the second application period, cause the energy delivery component to transmit the electrosurgical energy at a third power level, the third power level lower than the second power level and the third application period comprising a point in time where the impedance of the tissue rises above a transition impedance threshold level.

In another aspect of the surgical system, the first application period and the second application period combined comprise a time period where the electrosurgical energy causes sealing of the tissue at the surgical site.

In another aspect of the surgical system, the third application period further comprises a time period where the impedance of the tissue rises to a level such that the electrosurgical energy no longer causes sealing of the tissue at the surgical site.

In another aspect, the surgical system further comprises at least one sensor configured to measure an initial level of impedance in the tissue and a minimum level of impedance in the tissue.

In another aspect of the surgical system, the control circuit is further programmed to determine a beginning of the third application period based on the measured initial level of impedance in the tissue.

In another aspect of the surgical system, the control circuit is further programmed to determine a beginning of the third application period based on the measured minimum level of impedance in the tissue.

In another aspect of the surgical system, the first application period and the second application period combined comprise a continuous time period where the tissue impedance remains below an initial level of impedance in the tissue.

In another aspect of the surgical system, the energy delivery component is configured to transmit RF and ultrasonic energy.

In other aspects, a method for transmitting electrosurgical energy to tissue at a surgical site by a surgical system is provided. The method may include: causing, by an energy delivery component of a surgical system, electrosurgical energy to be applied to the tissue; measuring, by at least one sensor of the surgical system, a benchmark level of impedance of the tissue; determining, among a plurality of power load curve algorithms, a power load curve algorithm to be applied to the energy delivery component, based on the measured benchmark level of impedance of the tissue; based on the determined power load curve algorithm: for a first application period, causing the energy delivery component to transmit the electrosurgical energy at a first power level, the first application period comprising a point in time where impedance of the tissue reaches a minimum value; for a second application period after the first application period, cause the energy delivery component to transmit the electrosurgical energy at a decreasing power level from the first power level until a second power level is reached, the second power level lower than the first power level and the second application period comprising a point in time where the impedance of the tissue rises above the minimum impedance value; for a third application period after the second application period, cause the energy delivery component to transmit the electrosurgical energy at a third power level, the third power level lower than the second power level and the third application period comprising a point in time where the impedance of the tissue rises above a transition impedance threshold level.

In other aspects of the method, determining the power load curve algorithm comprises determining whether the benchmark level of impedance is less than a first threshold impedance value, whether the benchmark level of impedance is greater than the first threshold impedance value and less than a second threshold impedance value, and whether the benchmark level of impedance is greater than the second threshold impedance value.

In other aspects of the method, the first application period and the second application period combined comprise a time period where the electrosurgical energy is delivered at a higher rate and causes sealing of the tissue at the surgical site.

In other aspects of the method, the third application period further comprises a time period where the impedance of the tissue rises to a level such that the electrosurgical energy is delivered at a lower rate and no longer causes sealing of the tissue at the surgical site.

In other aspects of the method, the benchmark level of impedance is the minimum impedance value or an initial level of impedance of the tissue.

In other aspects of the method, a beginning of the third application period is based on the measured benchmark level of impedance.

In other aspects of the method, the first application period and the second application period combined comprise a continuous time period where the tissue impedance remains below an initial level of impedance in the tissue.

In other aspects of the method, the energy delivery component is configured to transmit RF and ultrasonic energy.

In other aspects, a surgical instrument is provided. The surgical instrument may include: a handle assembly; a shaft coupled to a distal end of the handle assembly; an end effector coupled to a distal end of the shaft and comprising at least one energy delivery component configured to transmit electrosurgical energy to tissue at a surgical site; and a control circuit communicatively coupled to the energy delivery component and programmed to: for a first application period, cause the energy delivery component to transmit the electrosurgical energy at a first energy level, the first application period comprising a point in time where impedance of the tissue reaches a minimum value; for a second application period after the first application period, cause the energy delivery component to transmit the electrosurgical energy at a decreasing energy level from the first energy level until a second energy level is reached, the second energy level lower than the first energy level and the second application period comprising a point in time where the impedance of the tissue rises above the minimum impedance value; for a third application period after the second application period, cause the energy delivery component to transmit the electrosurgical energy at a third energy level, the third energy level lower than the second energy level and the third application period comprising a point in time where the impedance of the tissue rises above a transition impedance threshold level.

In another aspect of the surgical instrument, the first application period and the second application period combined comprise a time period where the electrosurgical energy causes sealing of the tissue at the surgical site.

In another aspect of the surgical instrument, the third application period further comprises a time period where the impedance of the tissue rises to a level such that the electrosurgical energy no longer causes sealing of the tissue at the surgical site.

In another aspect, the surgical instrument further comprises at least one sensor configured to measure an initial level of impedance in the tissue and a minimum level of impedance in the tissue.

In other aspects, a non-transitory computer readable medium is presented. The computer readable medium may include instructions that, when executed by a processor, cause the processor to perform operations comprising any of the operations described in any one of the previous aspects.

The foregoing summary is illustrative only and is not intended to be in any way limiting. In addition to the illustrative aspects and features described above, further aspects and features will become apparent by reference to the drawings and the following detailed description.

### BRIEF DESCRIPTION OF THE DRAWINGS

The novel features of the aspects described herein are set forth with particularity in the appended claims. The aspects, however, both as to organization and methods of operation may be better understood by reference to the following description, taken in conjunction with the accompanying drawings as follows.

FIG. 1A shows one example of a surgical instrument system, according to some aspects.

FIG. 1B shows another example of a surgical instrument system, in this case showing multiple versions of a surgical instrument configured to deliver RF energy, ultrasonic energy, or a combination of both, according to some aspects.

FIG. 1C is a side, partially transparent schematic view of one aspect of a powered surgical device.

FIG. 1D is a side, partially transparent schematic view of another aspect of a powered surgical device.

FIG. 2 shows a perspective view of an end effector with jaws open, according to some aspects.

FIG. 3 is a block diagram describing further details of power supply elements of a surgical system, the surgical instrument coupled to a generator, according to some aspects.

FIG. 4A provides a visual depiction of the level of impedance over time present in tissue undergoing a sealing procedure during surgery.

FIG. 4B provides further details of various electrical readings of the surgical instrument system undergoing the sealing procedure during surgery.

FIG. 5 provides another example of the level of tissue impedance over time, this time using more empirical data.

FIG. 6A illustrates an example power profile of an amount of electrosurgical energy applied by a surgical instrument to tissue at a surgical site over time, in order to extend or prolong the bathtub region, according to some aspects.

FIG. 6B shows an example adjusted impedance profile over time as a result of the adjusted power applied to the surgical instrument, such as the example power profile in FIG. 6A, according to some aspects.

FIG. 7 shows an example power profile of the tapered load curve concept introduced in FIG. 6A, with additional power characteristics superimposed.

FIG. 8 provides an example of how multiple load curves may be programmed into the surgical instrument to apply different power adjustments based on impedance measurements during the sealing procedures.

FIG. 9 shows a visual depiction of an example load curve under the low minimum impedance threshold (see FIG. 8), according to some aspects.

FIG. 10 shows a visual depiction of an example load curve under the medium minimum impedance threshold (see FIG. 8), according to some aspects.

FIG. 11 is a graphical representation of impedance threshold and minimum pulse duration showing an example of additional adjustments that can be made to varying the power to account for other tissue properties.

FIG. 12 is a graphical illustration of voltage cutback caused by an impedance threshold greater than 325 Ohms, according to one aspect of the present invention.

FIG. 13 is a graphical illustration of a power pulse region of the graphical illustration shown in FIG. 11, according to one aspect of the present disclosure.

### DETAILED DESCRIPTION

In the following detailed description, reference is made to the accompanying drawings, which form a part hereof. In the drawings, similar symbols and reference characters typically identify similar components throughout the several views, unless context dictates otherwise. The illustrative aspects described in the detailed description, drawings, and claims are not meant to be limiting. Other aspects may be utilized, and other changes may be made, without departing from the scope of the subject matter presented here.

The following description of certain examples of the technology should not be used to limit its scope. Other examples, features, aspects, aspects, and advantages of the technology will become apparent to those skilled in the art from the following description, which is, by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, aspects, examples, etc. described herein may be combined with any one or more of the other teachings, expressions, aspects, examples, etc. that are described herein. The following described teachings, expressions, aspects, examples, etc. should, therefore, not be viewed in isolation relative to each other. Various suitable ways in which the teachings herein may be combined will be readily apparent to those of ordinary skill in the art in view of the teachings herein. Such modifications and variations are intended to be included within the scope of the claims.

Also, in the following description, it is to be understood that terms such as front, back, inside, outside, upper, lower, and the like are words of convenience and are not to be construed as limiting terms. Terminology used herein is not meant to be limiting insofar as devices described herein, or portions thereof, may be attached or utilized in other orientations. The various aspects will be described in more detail with reference to the drawings. Throughout this disclosure, the term “proximal” is used to describe the side of a component, e.g., a shaft, a handle assembly, etc., closer to a user operating the surgical instrument, e.g., a surgeon, and the term “distal” is used to describe the side of the component farther from the user operating the surgical instrument.

Aspects of the present disclosure are presented for a medical instrument configured to adjust the power level for sealing procedures to account for changes in tissue impedance levels over time. Typically during sealing procedures, a period of time exists where the tissue impedance level is low enough to allow for the tissue to respond to energy applied to it. The impedance level typically dips slightly over time initially, and then rises. After a certain point, due to various heating and chemical factors, the level of impedance rises dramatically, and energy applied to the tissue is no longer effective. Further example details of the limits of any power sources over a range of loads are described in some of the accompanying figures, below. The period of time where the level of impedance is low enough for applied energy to be effective is sometimes referred to as the “bathtub” region, due to the initial dip in the level of impedance and subsequent slow rise. It is desirable to manipulate the level of power applied to the tissue in order to extend or prolong the length of this bathtub region, so that the period of time for sealing and manipulating the tissue may be extended.

In some aspects, a medical instrument may be configured to apply power according to a power algorithm to seal tissue by applying a gradually lower amount of power over to time as the tissue impedance level begins to rise out of the “bathtub region.” In some aspects, the power is then cut once the tissue impedance level exceeds the “bathtub region.” By gradually reducing the power, a balance is achieved between still applying an effective level of power for sealing and prolonging the time in which the tissue impedance remains in the “bathtub region,” due to the reduced power.

The medical instrument of the present disclosures may include additional features. An end effector of the electrosurgical device may include multiple members arranged in various configurations to collectively perform the aforementioned functions. As used herein, an end effector may be referred to as a jaw assembly or clamp jaw assembly comprising an upper jaw member and a lower jaw member where at least one of the upper jaw member and the lower jaw member may be movable relative to the other. Each of the jaw members may be adapted to connect to an electrosurgical energy source. Each jaw member may incorporate an electrode. The electrode may be a positive or negative electrode. In a bipolar electrosurgical device, the electrodes may be adapted for connection to the opposite terminals of the electrosurgical energy source, such as a bipolar radio frequency (RF) generator, so as to generate a current flow therebetween. An electrosurgical energy may be selectively communicated through tissue held between the jaw members to effect a tissue seal and/or treatment. Tissue may be coagulated from the current flowing between the opposite polarity electrodes on each jaw member.

At least one jaw member may include a knife channel defined therein configured to reciprocate a knife there along for severing tissue held between the jaw members. The knife channel may be an extended slot in the jaw member. The knife may be provided within a recess associated with the at least one jaw member. The electrosurgical device may have both coagulation and cutting functions. This may eliminate or reduce instrument interchange during a surgery. Cutting may be achieved using mechanical force alone or a combination of mechanical force and the electrosurgical energy. The electrosurgical energy may be selectively used for coagulation and/or cutting. The knife may be made from an electrically conductive material adapted to connect to the electrosurgical source, and selectively activatable to separate tissue disposed between the jaw members. The knife may be spring biased such that once tissue is severed, the knife may automatically return to an unengaged position within the knife channel or a retracted position in the recess.

In some aspects, the jaw members may be movable relative to each other. During operation of the electrosurgical device, at least one of the jaw members may move from a first, open position where the jaw members can be disposed around a mass of tissue, to a second, closed position where the jaw members grasp the tissue. The jaw members therefore may move through a graspers-like range of motion, similar to that of conventional pliers. In the second position, current flows between the jaw members to achieve hemostasis of the tissue captured therebetween. The jaw members may be configured to have a relatively thick proximal portion to resist bending. At least one of the jaw members may have a three-dimensional configuration with a D-shaped cross-sectional. The three-dimensional configuration with the D-shaped cross-sectional may resist bending. A lock mechanism may be included to lock the jaw members in the closed position. The lock mechanism may set the clamp pressure between the jaw members. At least one electrically conductive gap setting member may be provided between the jaw members to establish a desired gap between electrodes in bipolar electrosurgical devices.

The electrosurgical device may incorporate components to grasp a tissue via the end effector, deliver energy to the tissue via one or more electrodes, and cut the tissue via a dissecting device such as a tissue knife. The structural capabilities of any aspect of an electrosurgical device may be designed for use in one or more of a variety of surgical procedures. In some surgical procedures, the treated tissue may be readily accessible to an end effector affixed to a relatively straight and unbendable shaft. In some alternative surgical procedures, the tissue may not be readily accessible to the end effector on such a shaft. In such procedures, the electrosurgical device may incorporate a shaft designed to bend so that the end effector may contact the tissue requiring treatment. In such a device, the shaft may include one or more articulated joints that may permit the shaft to bend under control by the user. A sliding knife may include a feature to provide actuating force to the sliding knife. A knife actuator may be operably coupled to the shaft for selectively reciprocating the knife through the knife channel.

A front portion assembly may be designed for a specific surgical procedure, while a reusable handle assembly, configured to releasably attach to a front portion assembly, may be designed to provide control of surgical functions common to each front portion assembly, such as tissue grasping, cauterizing, and cutting. Consequently, the number and types of devices required for surgeries can be reduced. The reusable handle assembly may be designed to automate common functions of the electrosurgical device. Device intelligence may be provided by a controller located in the reusable handle assembly that is configured to receive information from a front portion assembly. Such information may include data regarding the type and use of the front portion assembly. Alternatively, information may include data indicative of the position and/or activation of control components (such as buttons or slides that can be manipulated) that may indicate what system functions should be activated and in what manner.

In some non-limiting examples, the controller may supply the RF current when the energy activation control is placed in an activating position by the user. In some alternative non-limiting examples, the controller may supply the RF current for a predetermined period of time once the energy activation control is placed in an activing position. In yet another non-limiting example, the controller may receive data related to the position of the jaws and prevent the RF current from being supplied to the one or more tissue cauterization power contacts if the jaws are not in a closed position.

In some aspects, any of the mentioned examples also may be configured to articulate along at least one axis through various means, including, for example, a series of joints, one or more hinges or flexure bearings, and one or more cam or pulley systems. Other features may include cameras or lights coupled to one or more of the members of the end effector, and various energy options for the surgical device.

The electrosurgical device can be configured to source energy in various forms including, without limitation, electrical energy, monopolar and/or bipolar RF energy, microwave energy, reversible and/or irreversible electroporation energy, and/or ultrasonic energy, heat energy, or any combination thereof, to the tissue of a patient either independently or simultaneously. The energy can be transmitted to the electrosurgical device by a power source in electrical communication with the electrosurgical device. The power source may be a generator. The power source may be connected to the electrosurgical device via a suitable transmission medium such as a cable. The power source may be separate from the electrosurgical device or may be formed integrally with the electrosurgical device to form a unitary electrosurgical system. In one non-limiting example, the power source may include one or more batteries located within a portion of the electrosurgical device. It may be understood that the power source may source energy for use on the tissue of the patient as well as for any other electrical use by other devices, including, without limitation, lights, sensors, communication systems, indicators, and displays, which operate in relation to and/or with the electrosurgical device to form an electrosurgical system. In some aspects, the power source may source energy for use in measuring tissue effects with an RF impedance measuring portion. The remaining sources of energy, such as ultrasonic energy, may then be controlled based on the measured tissue effects. Similarly, multiple types of energy from one or more sources may be combined to interact in distinct ways. For example, an instrument with both RF and ultrasonic capabilities may allow for different energy types to perform different functions during a single procedure. For example, RF energy may be used to seal, while other energy types, such as ultrasonic energy, may be used to cut the tissue. In general, the present disclosures may be applied to these devices with combination elements (e.g., instruments having both RF and ultrasonic energy functionalities), and aspects are not so limited. Similar concepts include the systems and methods described in U.S. Pat. No. 9,017,326, IMPEDANCE MONITORING APPARATUS, SYSTEM, AND METHOD FOR ULTRASONIC SURGICAL INSTRUMENTS, which is incorporated herein by reference.

The electrosurgical device may be configured to source electrical energy in the form of RF energy. The electrosurgical device can transmit the RF energy through tissue compressed between two or more jaws. Such RF energy may cause ionic agitation in the tissue, in effect producing resistive heating, and thereby increasing the temperature of the tissue. Increased temperature of the tissue may lead to tissue cauterization. In some surgical procedures, RF energy may be useful for removing, shrinking, or sculpting soft tissue while simultaneously sealing blood vessels. RF energy may work particularly well on connective tissue, which is primarily composed of collagen and shrinks when contacted by heat. Because a sharp boundary may be created between the affected tissue and the surrounding tissue, surgeons can operate with a high level of precision and control, without sacrificing untargeted adjacent tissue.

The RF energy may be in a frequency range described in EN 60601-2-2:2009+A11:2011, Definition 201.3.218—HIGH FREQUENCY. For example, the frequency in monopolar RF applications may be typically restricted to less than 5 MHz. However, in bipolar RF applications, the frequency can be almost anything. Frequencies above 200 kHz can be typically used for monopolar applications in order to avoid the unwanted stimulation of nerves and muscles that would result from the use of low frequency current. Lower frequencies may be used for bipolar applications if the risk analysis shows the possibility of neuromuscular stimulation has been mitigated to an acceptable level. Normally, frequencies above 5 MHz are not used in order to minimize the problems associated with high frequency leakage currents. Higher frequencies may, however, be used in the case of bipolar applications. It is generally recognized that 10 mA is the lower threshold of thermal effects on tissue.

As discussed above, the electrosurgical device may be used in conjunction with a generator. The generator may be an electrosurgical generator characterized by a fixed internal impedance and fixed operating frequency that deliver maximum power to an external load (e.g., tissue), such as having an electrical impedance in the range of about 50 ohms to 150 ohms. In this type of bipolar electrosurgical generator, the applied voltage may increase monotonically as the load impedance increases toward the maximum “open circuit” voltage as the load impedance increases to levels of tens of thousands of ohms or more. In addition, the electrosurgical device may be used with a bipolar electrosurgical generator having a fixed operating frequency and an output voltage that may be substantially constant over a range of load impedances of tens of ohms to tens of thousands of ohms including “open circuit” conditions. The electrosurgical device may be advantageously used with a bipolar electrosurgical generator of either a variable voltage design or substantially constant voltage design in which the applied voltage may be interrupted when the delivered current decreases below a predetermined level. Such bipolar generators may be referred to as automatic generators in that they may sense the completion of the coagulation process and terminate the application of voltage, often accompanied by an audible indication in the form of a cessation of a “voltage application” tone or the annunciation of a unique “coagulation complete” tone. Further, the electrosurgical device may be used with an electrosurgical generator whose operating frequency may vary with the load impedance as a means to modulate the applied voltage with changes in load impedance.

Various aspects of electrosurgical devices use therapeutic and/or sub-therapeutic electrical energy to treat tissue. Some aspects may be utilized in robotic applications. Some aspects may be adapted for use in a hand operated manner. In one non-limiting example, an electrosurgical device may include a proximal handle, a distal working end or end effector, and an introducer or elongated shaft disposed in-between.

Additional details regarding electrosurgical end effectors, jaw closing mechanisms, and electrosurgical energy-delivery surfaces are described in the following U.S. patents and published patent applications: U.S. Pat. Nos. 7,087,054; 7,083,619; 7,070,597; 7,041,102; 7,011,657; 6,929,644; 6,926,716; 6,913,579; 6,905,497; 6,802,843; 6,770,072; 6,656,177; and 6,533,784; and U.S. Patent Application Publication Nos. 2010/0036370 and 2009/0076506, all of which are incorporated herein by reference in their entirety and made part of this specification.

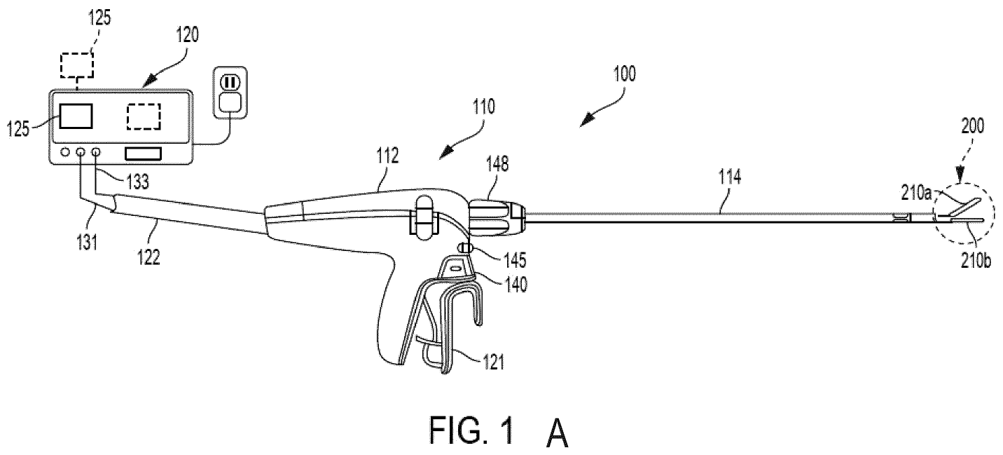

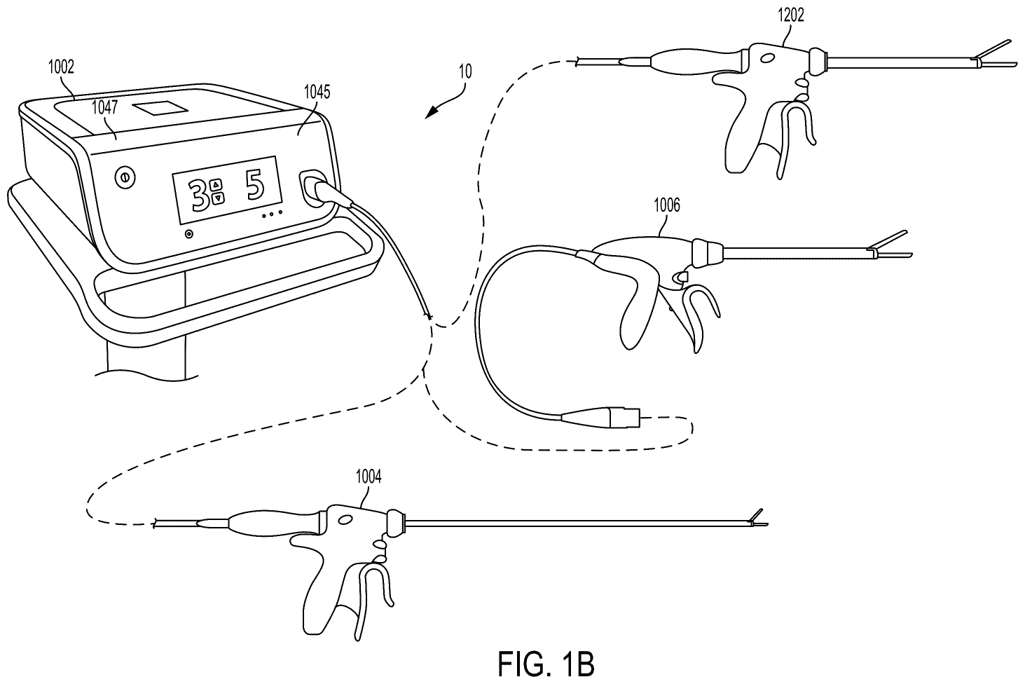

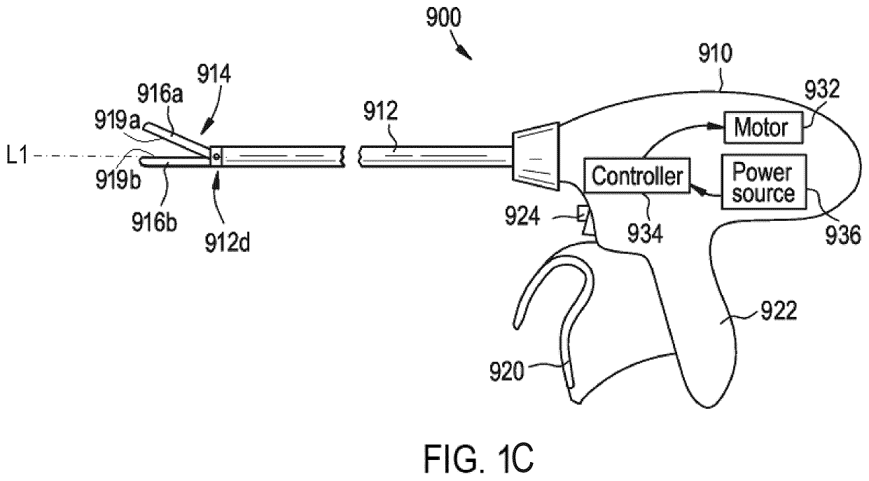

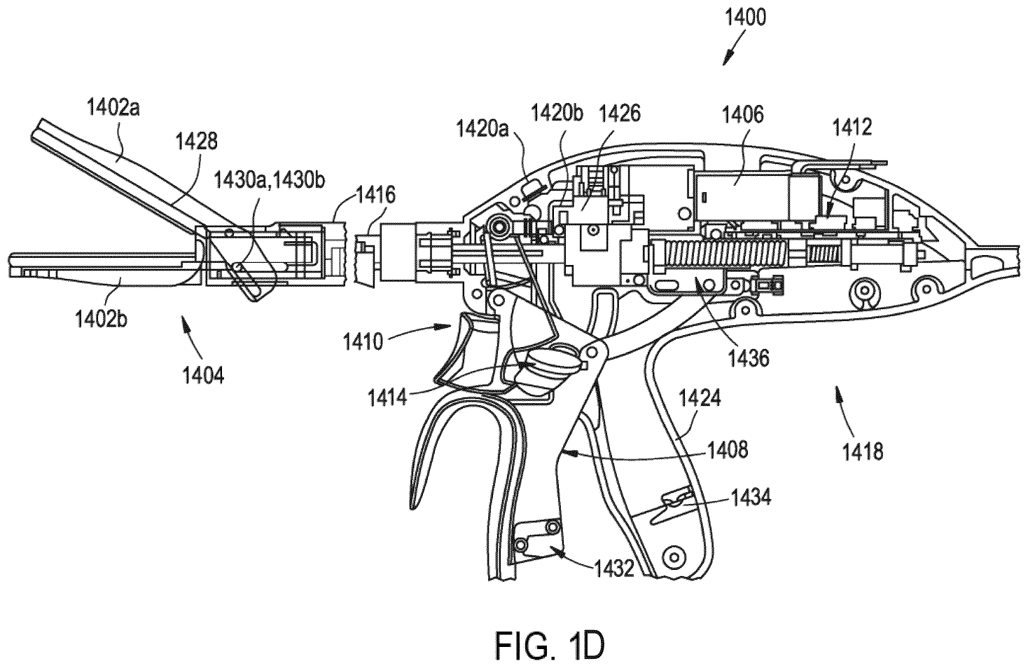

FIG. 1A shows one example of a surgical instrument system 100, according to aspects of the present disclosure. The surgical instrument system 100 comprises an electrosurgical instrument 110. The electrosurgical instrument 110 may comprise a proximal handle 112, a distal working end or end effector 200 and an introducer or elongated shaft 114 disposed in-between. Alternatively, the end effector may be attached directly to the handle as in scissor style devices such as the electrosurgical instrument described in U.S. Pat. No. 7,582,087.

The electrosurgical system 100 can be configured to supply energy, such as electrical energy, ultrasonic energy, heat energy, or any combination thereof, to the tissue of a patient either independently or simultaneously, for example. In one example, the electrosurgical system 100 may include a generator 120 in electrical communication with the electrosurgical instrument 110. The generator 120 may be connected to the electrosurgical instrument 110 via a suitable transmission medium such as a cable 122. In one example, the generator 120 may be coupled to a controller, such as a control unit 125, for example. In various aspects, the control unit 125 may be formed integrally with the generator 120 or may be provided as a separate circuit module or device electrically coupled to the generator 120 (shown in phantom to illustrate this option). The control unit 125 may include automated or manually operated controls to control the amount of current delivered by the generator 120 to the electrosurgical instrument 110. Although as presently disclosed, the generator 120 is shown separate from the electrosurgical instrument 110, in some aspects, the generator 120 (and/or the control unit 125) may be formed integrally with the electrosurgical instrument 110 to form a unitary electrosurgical system 100, where a battery located within the electrosurgical instrument 110 may be the energy source and a circuit coupled to the battery produces the suitable electrical energy, ultrasonic energy, or heat energy.

In one aspect, the generator 120 may comprise an input device located on a front panel of the generator 120 console. The input device may comprise any suitable device that generates signals suitable for programming the operation of the generator 120, such as a keyboard, or input port, for example. In one example, one or more electrodes in the first jaw 210a and one or more electrodes in the second jaw 210b may be coupled to the generator 120. The cable 122 may comprise multiple electrical conductors for the application of electrical energy to a first electrode (which may be designated as a +electrode) and to a second electrode (which may be designated as a −electrode) of the electrosurgical instrument 110. It may be recognized that + and − designations are made solely for convenience and do not indicate an electrical polarity. An end of each of the conductors may be placed in electrical communication with a terminal of the generator 120. The generator 120 may have multiple terminals, each configured to contact one or more of the conductors. The control unit 125 may be used to activate the generator 120, which may serve as an electrical source. In various aspects, the generator 120 may comprise an RF source, an ultrasonic source, a direct current source, and/or any other suitable type of electrical energy source, for example, which may be activated independently or simultaneously.

In various aspects, the electrosurgical system 100 may comprise at least one supply conductor 131 and at least one return conductor 133, wherein current can be supplied to the electrosurgical instrument 110 via the at least one supply conductor 131 and wherein the current can flow back to the generator 120 via the at least one return conductor 133. In various aspects, the at least one supply conductor 131 and the at least one return conductor 133 may comprise insulated wires and/or any other suitable type of conductor. As described below, the at least one supply conductor 131 and the at least one return conductor 133 may be contained within and/or may comprise the cable 122 extending between, or at least partially between, the generator 120 and the end effector 200 of the electrosurgical instrument 110. The generator 120 can be configured to apply a sufficient voltage differential between the supply conductor 131 and the return conductor 133 such that sufficient current can be supplied to the end effector 200 to perform the intended electrosurgical operation.

The shaft 114 may have a cylindrical or rectangular cross-section, for example, and can comprise a thin-wall tubular sleeve that extends from the proximal handle 112. The shaft 114 may include a bore extending therethrough for carrying actuator mechanisms, for example, an axially moveable member for actuating the jaws 210a, 210b and for carrying electrical leads for delivery of electrical energy to electrosurgical components of the end effector 200. The proximal handle 112 may include a jaw closure trigger 121 configured to adjust the position of the jaws 210a, 210b with respect to each other. In one non-limiting example, the jaw closure trigger 121 may be coupled to an axially moveable member disposed within the shaft 114 by a shuttle operably engaged to an extension of the jaw closure trigger 121.

The end effector 200 may be adapted for capturing and transecting tissue and for contemporaneously welding the captured tissue with controlled application of energy (e.g., RF energy). The first jaw 210a and the second jaw 210b may be closed thereby capturing or engaging tissue. The first jaw 210a and second jaw 210b also may apply compression to the tissue. In some aspects, the shaft 114, along with the first jaw 210a and second jaw 210b, can be rotated, for example, a full 360°. For example, a rotation knob 148 may be rotatable about the longitudinal axis of the shaft 114 and may be coupled to the shaft 114 such that rotation of the knob 148 causes corresponding rotation of the shaft 114. The first jaw 210a and the second jaw 210b can remain openable and/or closeable while rotated.

Also illustrated in FIG. 1 are a knife advancement control 140 and an energy activation control 145 located on the proximal handle 112. In some non-limiting examples, the knife advancement control 140 and the energy activation control 145 may be depressible buttons positioned to permit a user to control knife advancement or energy activation by the use of one or more fingers.

FIG. 1B illustrates a second example of a surgical system 10 comprising a generator 1002 and various surgical instruments 1004, 1006, 1202 usable therewith, according to some aspects. The generator 1002 may be configurable for use with a variety of surgical devices. According to various forms, the generator 1002 may be configurable for use with different surgical devices of different types including, for example, the ultrasonic device 1004, electrosurgical or RF surgical devices, such as, the RF device 1006, and multifunction devices 1202 that integrate electrosurgical RF and ultrasonic energies delivered simultaneously from the generator 1002. Although in the form of FIG. 1B, the generator 1002 is shown separate from the surgical devices 1004, 1006, 1202 in one form, the generator 1002 may be formed integrally with either of the surgical devices 1004, 1006, 1202 to form a unitary surgical system. The generator 1002 comprises an input device 1045 located on a front panel of the generator 1002 console. The input device 1045 may comprise any suitable device that generates signals suitable for programming the operation of the generator 1002.

FIG. 1C illustrates one aspect of a surgical device 900 configured to grasp and cut tissue. The surgical device 900 can include a proximal handle portion 910, a shaft portion 912, and an end effector 914 configured to grasp tissue. The proximal handle portion 910 can be any type of pistol-grip or other type of handle known in the art that is configured to carry various actuators, such as actuator levers, triggers or sliders, configured to actuate the end effector 914. As illustrated, the proximal handle portion 910 can include a closure grip 920 and a stationary grip 922. Movement of the closure grip 920 toward and away from the stationary grip 922, such as by manual movement by a hand of a user, can adjust a position of the end effector 914. The shaft portion 912 can extend distally from the proximal handle portion 910 and can have a bore (not shown) extending therethrough. The bore can carry mechanisms for actuating the end effector 914, such as a jaw closure tube and/or a drive shaft. As discussed further below, one or more sensors (not shown) can be coupled to the surgical device 900 and can be configured to sense data that can be used in controlling an output of the device's motor 932.

The end effector 914 can have a variety of sizes, shapes, and configurations. As shown in FIG. 1C, the end effector 914 can include a first, upper jaw 16a and a second, lower jaw 916b each disposed at a distal end 912d of the shaft portion 912. One or both of the upper and lower jaws 916a, 916b can be configured to close or approximate about a longitudinal axis L1 of the end effector 914. Both of the jaws 916a, 916b can be moveable relative to the shaft portion 912 such that the end effector 914 can be moved between open and closed positions, or only one the upper and lower jaws 916a, 916b can be configured to move relative to the shaft portion 912 and to the other of the jaws 916a, 916b so as to move the end effector 914 between open and closed positions. When the end effector 914 is in the open position, the jaws 916a, 916b can be positioned at a distance apart from one another with space therebetween. As discussed further below, tissue can be positioned within the space between the jaws 916a, 916b. When the end effector 914 is in the closed position, a longitudinal axis of the upper jaw 916a can be substantially parallel to a longitudinal axis of the lower jaw 916b, and the jaws 916a, 916b can be moved toward one another such that the distance therebetween is less than when the end effector 914 is in the open position. In some aspects, facing engagement surfaces 919a, 919b of the jaws 916a, 916b can be in direct contact with one another when the end effector 914 is in the closed position such that the distance between is substantially zero. As illustrated, the upper jaw 16a is configured to pivot relative to the shaft portion 912 and relative to the lower jaw 916b while the lower jaw 916b remains stationary. As illustrated, the jaws 916a, 916b have a substantially elongate and straight shape, but a person skilled in the art will appreciate that one or both of the jaws 916a, 916b can be curved along the longitudinal axis L1 of the end effector 914. The longitudinal axis L1 of the end effector 914 can be parallel to and coaxial with a longitudinal axis of the shaft portion 912 at least when the end effector 914 is in the closed configuration, and if the end effector 914 is configured to articulate relative to the shaft portion 912, when the end effector 914 is not articulated relative to the shaft portion 912.

FIG. 1D illustrates another aspect of a surgical device 1400 configured to cut and seal tissue clamped between first and second jaws 1402a, 1402b of the device's end effector 1404. The device 1400 can be configured to separately cut and seal tissue and configured to simultaneously cut and seal tissue, with a user of the device 1400 being able to decide whether cutting and sealing occurs separately or simultaneously. The device 1400 can generally be configured similar to the device 900 of FIG. 1C. The device 1400 can include a motor 1406, a closure trigger 1408, a firing actuator 1410, a controller 1412, a cutting element (not shown), a power connector (not shown) configured to attach to an external power source (not shown), an energy actuator 1414, an elongate shaft 1416 extending from a handle portion 1418 of the device 1400, a sensor 1420a, 1420b, the end effector 1404 at a distal end of the shaft 1416, a stationary handle 1424, and a gear box 1426 that can be operatively connected to the motor 1406 and configured to transfer output from the motor 1406 to the cutting element. As illustrated, the controller 1412 includes a printed circuit board (PCB), the sensor 1420a includes a Hall effect sensor, and the other sensor 1420b includes a Hall effect sensor. One of the jaws 1420a includes an insulator 1428 configured to facilitate safe energy application to tissue clamped by the end effector 1404. Each of the jaws 1402a, 1402b can include a proximal slot 1430a, 1430b configured to facilitate opening and closing of the end effector 1404, as will be appreciated by a person skilled in the art. The device 1400 can be configured to lock the closure trigger 1408 in the closed position, such as by the closure trigger 1408 including a latch 1432 configured to engage a corresponding latch 1434 on the stationary handle 1424 when the closure trigger 1408 is drawn close enough thereto so as to lock the closure trigger 1408 in position relative to the stationary handle 1424. The closure trigger latch 1432 can be configured to be manually released by a user so as to unlock and release the closure trigger 1408. A bias spring 1436 included in the handle portion 1418 can be coupled to the closure trigger 1408 and cause the closure trigger 1408 to open, e.g., move away from the stationary handle 1424, when the closure trigger 1408 is unlocked.

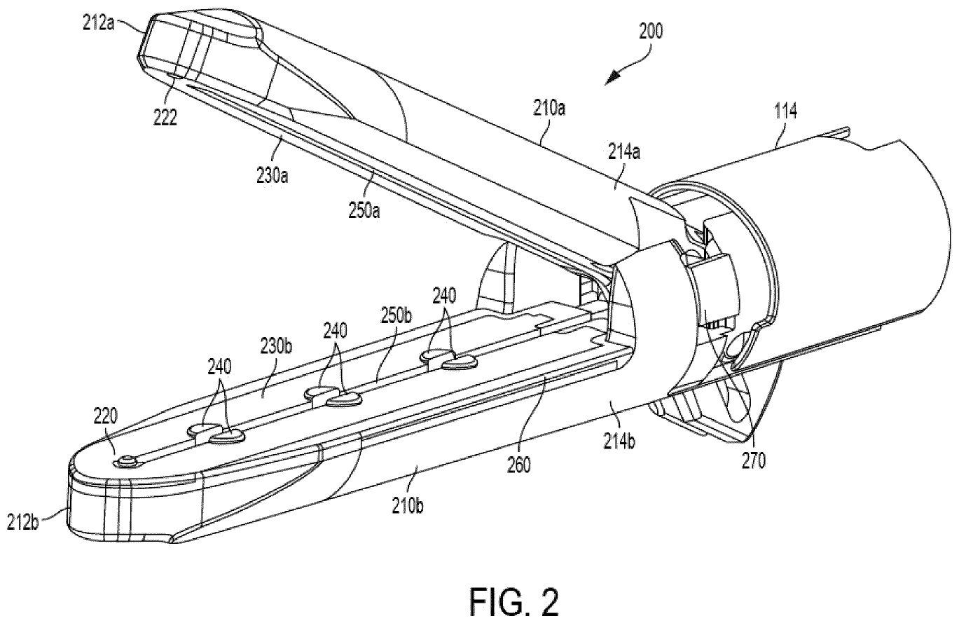

FIG. 2 shows a perspective view of the end effector 200 with the jaws 210a, 210b open, according to one aspect of the present disclosure. The end effector 200 may be attached to any of the various surgical instruments provided herein, including those configured to supply RF energy, ultrasonic energy, or various combinations of energy to the end effector 200. The end effector 200 may comprise the first or upper jaw 210a and the second or lower jaw 210b, which may be straight or curved. The upper jaw 210a may comprise a first distal end 212a and a first proximal end 214a. The lower jaw 210b may comprise a second distal end 212b and a second proximal end 214b. The first distal end 212a and the second distal end 212b may be collectively referred to as the distal end of the end effector when the jaws 210a, 210b are in a closed configuration. The first proximal end 214a and the second proximal end 214b may be collectively referred to as the proximal end of the end effector 200. The jaws 210a, 210b are pivotally coupled at the first and second proximal ends 214a, 214b. As shown in FIG. 2, The lower jaw 210b is fixed and the upper jaw 210a is pivotally movable relative to the lower jaw 210b from an open position to a closed position and vice versa. In the closed position, the first and second distal ends 212a, 212b are in proximity. In the open position, the first and second distal ends 214a, 214b are spaced apart. In other aspects, the upper jaw 210a may be fixed and the lower jaw 210b may be movable. In other aspects, both the upper and lower jaws 210a, 210b may be movable. The end effector 200 may comprise a pivot assembly 270 located at or in proximity to the proximal end of the end effector, which sets an initial gap between the jaws 210a, 210b at the proximal end of the end effector 200 in a closed position. The pivot assembly 270 may be welded in place in a gap setting process during manufacturing of the end effector 200, as described in greater detail below.

In some aspects, the first jaw 210a and the second jaw 210b may each comprise an elongated slot or channel 250a and 250b, respectively, disposed along their respective middle portions. The channels 250a and 250b may be sized and configured to accommodate the movement of an axially moveable member (not shown), which may comprise a tissue-cutting element, for example, comprising a sharp distal edge. The upper jaw 210a may comprise a first energy delivery surface 230a. The lower jaw 210b may comprise a second energy delivery surface 230b. The first and second energy delivery surfaces 230a, 230b face each other when the jaws 210a, 210b are in a closed configuration. The first energy delivery surface 230a may extend in a “U” shape around the channel 250a, connecting at the first distal end 212a. The second energy delivery surface 230b may extend in a “U” shape around the channel 250b, connecting at the second distal end 212b. The first and second energy delivery surfaces 230a, 230b may comprise electrically conductive material such as copper, gold plated copper, silver, platinum, stainless steel, aluminum, or any suitable electrically conductive biocompatible material, for example. The second energy delivery surface 230b may be connected to the supply conductor 131 shown in FIG. 1A, for example, thus forming the first electrode in the electrosurgical instrument 110. The first energy delivery surface 230a may be connected and the return conductor 133 shown in FIG. 1A, thus forming the second electrode the electrosurgical instrument 110. For example, the first energy delivery surface 230a may be grounded. Opposite connection is also possible.

As shown in FIG. 2, the second energy delivery surface 230b is formed by a conductive layer disposed, or at least partially disposed, along at least a portion of the body of the lower jaw 210b. The electrically conductive layer comprising the second energy delivery surface 230b may extend to the second distal end 212b, and thus operation of the end effector 200 is possible without actually grasping the tissue. The lower jaw 210b may further comprise an electrically insulative layer 260 arranged between the conductive layer and the body of the lower jaw 210b. The electrically insulative layer 260 may comprise electrically insulative material such as ceramic or nylon. Furthermore, the first energy delivery surface 230a is formed of an electrically conductive layer disposed, or at least partially disposed, along at least a portion of the body of the upper jaw 210a. The upper jaw 210a also may comprise an electrically insulative layer arranged between the conductive layer and the body of the upper jaw 210a.

The upper and lower jaws 210a and 210b may each comprise one or more electrically insulative tissue engaging members arranged on the first or second energy delivery surface 230a, 230b, respectively. Each of the electrically insulative tissue engaging members may protrude from the energy delivery surface and define a height above the energy delivery surface, and thus is sometimes referred to as a “tooth.” The electrically insulative tissue engaging members may comprise electrically insulative material such as ceramic or nylon. As shown in FIG. 2, the electrically insulative tissue engaging members 240 are arranged longitudinally, e.g., along the length of the lower jaw 210b, from the send proximal end 214b to the second distal end 212b and on the second energy delivery surface 230b. As shown in FIG. 2, the electrically insulative tissue engaging members 240 are in pairs, and each pair is arranged next to the channel 250b and is separated by the channel 250b. These paired electrically insulative tissue engaging members 240 as arranged here are sometimes referred to as “teeth.”

In other aspects, other configurations of the electrically insulative tissue engaging members 240 are possible. For example, the electrically insulative tissue engaging members 240 may be located at a distance away from the channel. For example, the electrically insulative tissue engaging members 240 may be arranged in a grid on the energy delivery surface. In other aspects, the electrically insulative tissue engaging members 240 may not have the half cylindrical cross sections as shown in FIG. 2, and may have cylindrical cross sections or rectangular cross sections. In other aspects, electrically insulative tissue engaging members 240 may be arranged on the first energy delivery surface 230a, or may be arranged on both of the first and second energy delivery surfaces 230a, 230b.

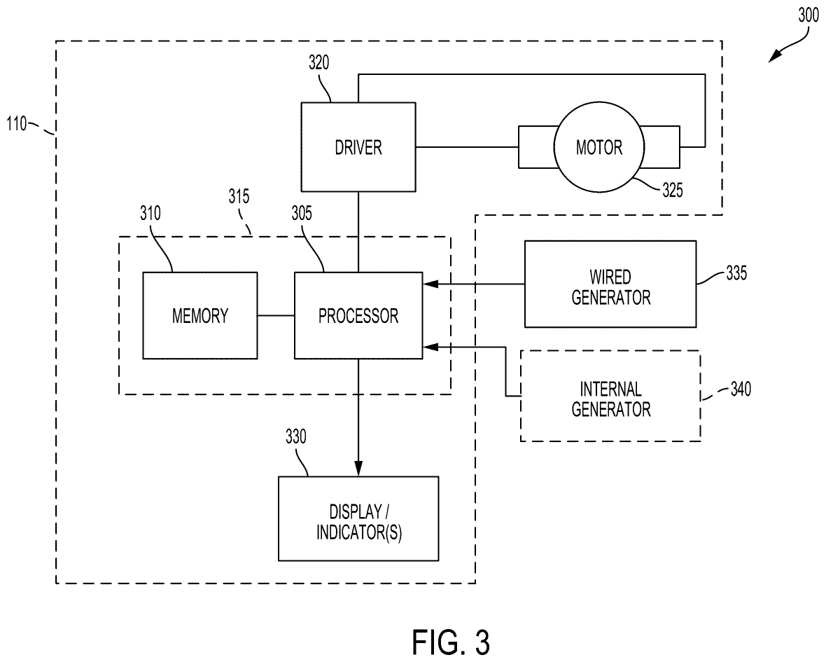

FIG. 3 is a block diagram of a surgical system 300 comprising a motor-driven surgical grasping instrument 900, 1400 (FIGS. 1C, 1D) according to some aspects of the present disclosure, the surgical instrument coupled to a generator 335 (340), according to some aspects. The motor-driven surgical cutting and fastening instrument 2 described in the present disclosure may be coupled to a generator 335 (340) configured to supply power to the surgical instrument through external or internal means. FIG. 3 describes examples of the portions for how electrosurgical energy may be delivered to the end effector. In certain instances, the motor-driven surgical instrument 110 may include a microcontroller 315 coupled to an external wired generator 335 or internal generator 340. Either the external generator 335 or the internal generator 340 may be coupled to A/C mains or may be battery operated or combinations thereof. The electrical and electronic circuit elements associated with the motor-driven surgical instrument 110 and/or the generator elements 335, 340 may be supported by a control circuit board assembly, for example. The microcontroller 315 may generally comprise a memory 310 and a microprocessor 305 (“processor”) operationally coupled to the memory 310. The microcontroller 315 may be configured to regulate the electrosurgical energy applied at the end effector according to the concepts disclosed herein and described more below. The processor 305 may control a motor driver 320 circuit generally utilized to control the position and velocity of the motor 325. The motor 325 may be configured to control transmission of energy to the electrodes at the end effector of the surgical instrument. In certain instances, the processor 305 can signal the motor driver 320 to stop and/or disable the motor 325, as described in greater detail below. In certain instances, the processor 305 may control a separate motor override circuit which may comprise a motor override switch that can stop and/or disable the motor 325 during operation of the surgical instrument in response to an override signal from the processor 305. It should be understood that the term processor as used herein includes any suitable microprocessor, microcontroller, or other basic computing device that incorporates the functions of a computer's central processing unit (CPU) on an integrated circuit or at most a few integrated circuits. The processor is a multipurpose, programmable device that accepts digital data as input, processes it according to instructions stored in its memory, and provides results as output. It is an example of sequential digital logic, as it has internal memory. Processors operate on numbers and symbols represented in the binary numeral system.

In some cases, the processor 305 may be any single core or multicore processor such as those known under the trade name ARM Cortex by Texas Instruments. In some cases, any of the surgical instruments of the present disclosures may comprise a safety processor such as, for example, a safety microcontroller platform comprising two microcontroller-based families such as TMS570 and RM4x known under the trade name Hercules ARM Cortex R4, also by Texas Instruments. Nevertheless, other suitable substitutes for microcontrollers and safety processor may be employed, without limitation. In one instance, the safety processor may be configured specifically for IEC 61508 and ISO 26262 safety critical applications, among others, to provide advanced integrated safety features while delivering scalable performance, connectivity, and memory options.

In certain instances, the microcontroller 315 may be an LM 4F230H5QR, available from Texas Instruments, for example. In at least one example, the Texas Instruments LM4F230H5QR is an ARM Cortex-M4F Processor Core comprising on-chip memory 310 of 256 KB single-cycle flash memory, or other non-volatile memory, up to 40 MHz, a prefetch buffer to improve performance above 40 MHz, a 32 KB single-cycle serial random access memory (SRAM), internal read-only memory (ROM) loaded with StellarisWare® software, 2 KB electrically erasable programmable read-only memory (EEPROM), one or more pulse width modulation (PWM) modules, one or more quadrature encoder inputs (QEI) analog, one or more 12-bit Analog-to-Digital Converters (ADC) with 12 analog input channels, among other features that are readily available for the product datasheet. Other microcontrollers may be readily substituted for use in the motor-driven surgical instrument 110. Accordingly, the present disclosure should not be limited in this context.

Referring again to FIG. 3, the surgical system 300 may include a wired generator 335, for example. In certain instances, the wired generator 335 may be configured to supply power through external means, such as through electrical wire coupled to an external generator. In some cases, the surgical system 300 also may include or alternatively include an internal generator 340. The internal generator 340 may be configured to supply power through internal means, such as through battery power or other stored capacitive source. Further descriptions of the internal generator 340 and the wired generator 335 are described below.

In certain instances, the motor-driven surgical instrument 110 may comprise one or more embedded applications implemented as firmware, software, hardware, or any combination thereof. In certain instances, the motor-driven surgical instrument 110 may comprise various executable modules such as software, programs, data, drivers, and/or application program interfaces (APIs), for example.

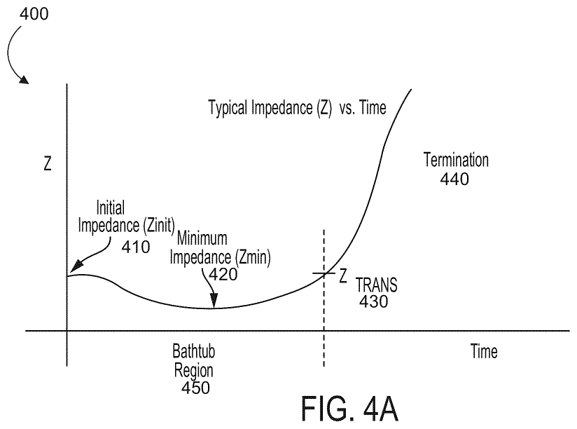

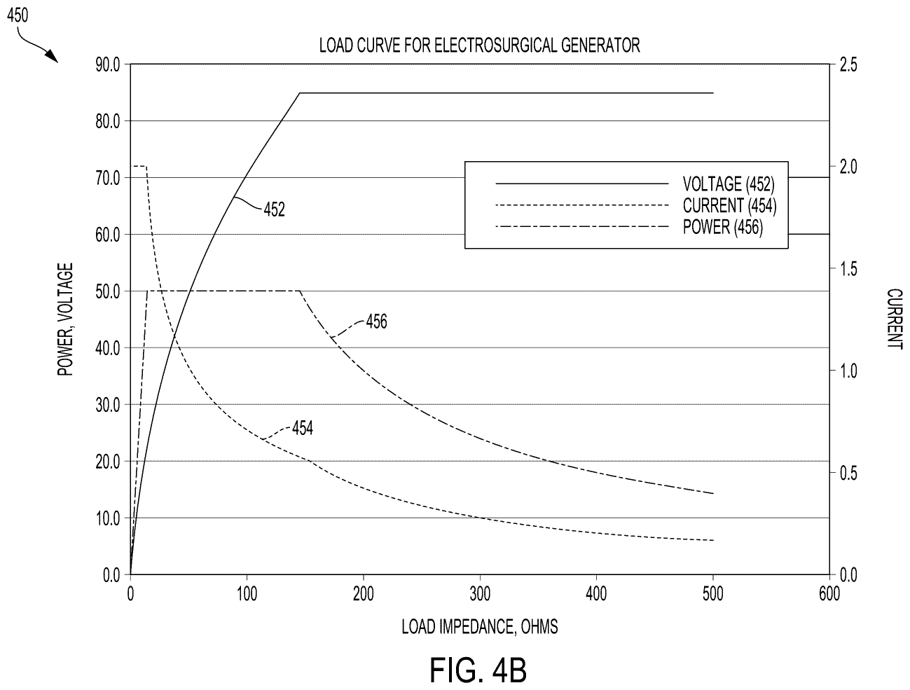

Referring to FIG. 4A, graph 400 and provides a visual depiction of the level of impedance over time present in tissue undergoing a sealing procedure during surgery. This example graph 400 provides a conceptual framework for the types of power adjustments employed according to the present disclosures. Here, time zero represents the first point at which a surgical instrument, such as instrument 110, applies electrosurgical energy to tissue at a surgical site. The Y axis represents the level of tissue impedance (Z) present when a substantially constant level of power is applied to the tissue via an end effector of the instrument 110. At time zero, the tissue exhibits an initial level of impedance (Zᵢₙᵢₜ) 410. The initial level of impedance 410 may be based on native physiological properties about the tissue, such as density, amount of moisture, and what type of tissue it is. Over a short period of time, it is known that the level of impedance actually dips slightly as power is continuously applied to the tissue. This is a common phenomenon that occurs in all kinds of tissue. A minimum level of impedance (Zₘᵢₙ) 420 is eventually reached. From here, the overall level of impedance monotonically increases, and at first increases with a slow rise. Eventually, a transition point is reached such that level of impedance starts to dramatically rise above the initial level of impedance 410. This point is generally known as a transition impedance level (Zₜᵣₐₙₛ) 430. After the transition impedance 430 is reached, the level of impedance rises dramatically over time, and beyond this point the tissue impedance is generally too high for electrosurgical energy to have a substantial impact on the tissue. Therefore, termination 440 of the electrosurgical energy generally occurs soon after the transition impedance 430 point is reached. Thus, the period of time between the initial impedance 410 and when the transition impedance 430 is reached is generally the only effective time when electrosurgical energy may be applied to the tissue with any positive effect. This region is sometimes known as the bathtub region 450, due to the general shape of the curve over this time period. It is therefore desirable extend or prolong the bathtub region 450 in order to increase the amount of time where electrosurgical energy may be applied.

Referring to FIG. 4B, graph 450 shows an example of a typical load curve for a generator configured to provide power to an electrosurgical system of the present disclosure. The left vertical axis represents power (W) and voltage (V), the right vertical axis represents current (A), and the horizontal axis represents load impedance (Ohms). The voltage curve 452, current curve 454, and power curve 456 are shown as functions of load impedance. As shown, the amount of power and voltage applied to tissue typically reaches an impassable threshold, even over ever increasing load impedances. Looked at another way, the amount of energy applied to the tissue at a surgical site has a noticeable effect only up to certain levels of load impedances, and after a certain impedance threshold, such as 175 ohms, applying more or sustained power typically has little to no benefit. Graph 450 therefore provides further detail on why exceeding the transition impedance threshold, as shown in graph 400, generally represents the cutoff point to which power should continue to be applied.

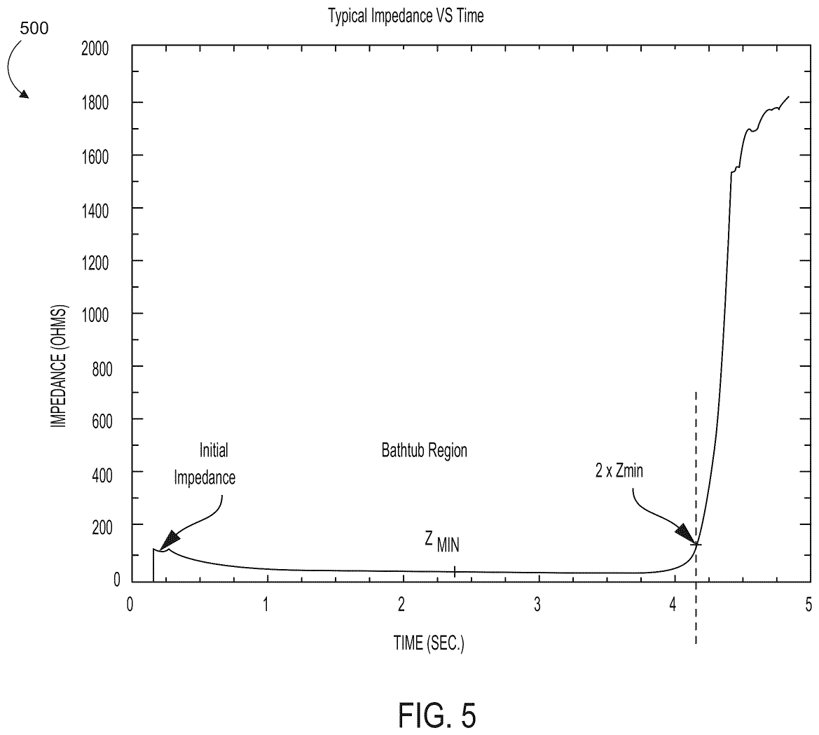

Referring to FIG. 5, graph 500 provides another example of the level of tissue impedance over time, this time using more empirical data. As shown, the level of tissue impedance stays relatively low until the transition impedance level is reached, which occurs a little after four seconds in this example. In some cases, the transition impedance level is defined by the point at which the level of impedance rises above twice the value of the minimum impedance level, while in other cases it may be defined as the point at which the initial impedance level is reached again. Regardless of which definition is used, the transition impedance level generally occurs at around the same time, assuming a constant level of power is applied to the tissue. Furthermore, it is known that the transition impedance level is reliably a function of the initial impedance level of the tissue. In other words, depending on what the initial level of impedance is, the transition impedance level can be predicted reliably in the tissue.

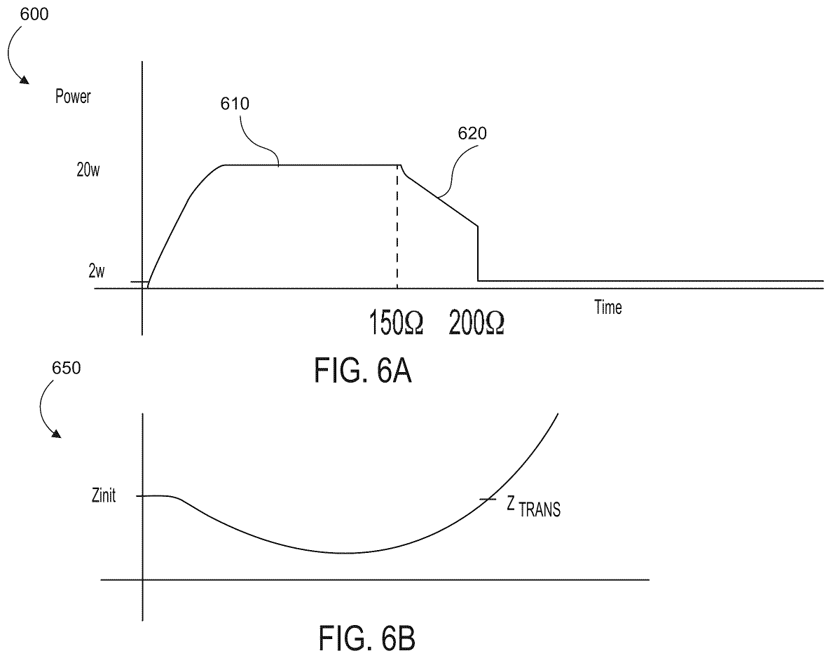

Referring to FIG. 6A, graph 600 illustrates an example power profile of an amount of electrosurgical energy applied by a surgical instrument 110 to tissue at a surgical site over time, in order to extend or prolong the bathtub region, according to some aspects. The continuous, piecewise curve 610 shows that the level of power changes in multiple stages. Initially, the electrosurgical energy applied rises to a predetermined level of power, such as 20 W in this case. This represents the initial desired level of power used for the surgical procedure in question. While the tissue impedance is generally in the bathtub region, this initial level of power is acceptable. However, as the tissue impedance begins to change and rises toward the transition impedance level, according to some aspects, the level of power is tapered down at a steady rate, rather than the level of power be constantly maintained like in conventional methods. In this example, it is supposed that the transition impedance level is 200 ohms, and may have been determined based on a measurement of the initial impedance level. Thus, as the tissue impedance rises but before it reaches the transition impedance level, such as when the tissue impedance reaches 75% of the transition impedance level (i.e., 150 ohms), the power system of the surgical instrument 110 may cause the level of power to decrease at a steady rate, as shown in the downward sloped region 620. For example, the power level may be dropped by 50% over the course of time where the tissue impedance level continues to climb until it reaches the transition impedance level. Finally, once the transition impedance level is reached, the power to the surgical instrument 110 may be cut dramatically, as further application of power beyond the transition impedance level may be ineffective or may even cause unwanted damage to the tissue. In some aspects, the tapering of the power may be achieved in a number of ways that are all within the purview of this disclosure. For example, the microprocessor 315 (see FIG. 3) may regulate the power coming from generators 335 or 340 via pulse width modulation or by applying an increase in resistance via the driver 320. In general, the present disclosures may employ any methods for lowering the power known to those with skill in the art, and aspects are not so limited.

Referring to FIG. 6B, graph 650 shows an example adjusted impedance profile over time as a result of the adjusted power applied to the surgical instrument 110, such as the example power profile 610, according to some aspects. As shown, the rise in impedance out of the bathtub region may be made more gradual due to the tapering of the power made according to aspects of the present disclosure. In addition, the overall length of the bathtub region may be extended or prolonged. The gradual rise of the tissue impedance may also allow for better care and treatment of the tissue under surgery. Conventionally, continuous power applied to the tissue after the transition impedance level is reached may cause unwanted damage to the tissue, such as burning tissue, sizzling, and popping. Due to the adjustments disclosed herein, this unwanted damage may be reduced or even eliminated.

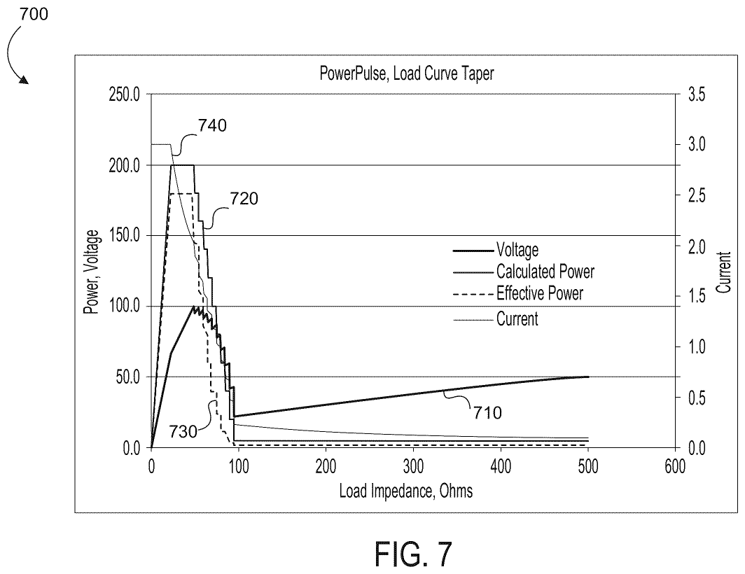

Referring to FIG. 7, graph 700 shows an example power profile of the tapered load curve concept introduced in FIG. 6A, with additional power characteristics superimposed. Here, the curve 710 as shown by the thick line represents a measure of voltage as a function of load impedance in the tissue. The curve 720 as shown by the medium line represents a measure of calculated power applied to the tissue as a function of load impedance. The calculated power may be the measure of power that is determined by the power system of the surgical instrument 110, while the curve 730 as shown by the dashed line represents the actual or effective power applied to the tissue. As shown, both of these curves exhibit a power taper that is reduced in a stepwise manner. This may be caused by the power being duty cycled at different rates over time, i.e., via pulse width modulation. The curve 740 as shown by the thin line represents a measure of current. The scale for the current is shown on the right-hand side, while the scale for power and voltage is shown on the left.

As shown, the power is tapered off in this example when the load impedance is measured to be approximately 60 to 70 ohms, and the power is nearly completely cut off when the load impedance is measured to be about 100 ohms. The voltage drops dramatically during the tapered region of the power profile, but begins to slowly rise again due to the low but constant application of power applied while the load impedance continues to increase. Practically, the power may be cut off long before the load impedance reaches these higher levels.

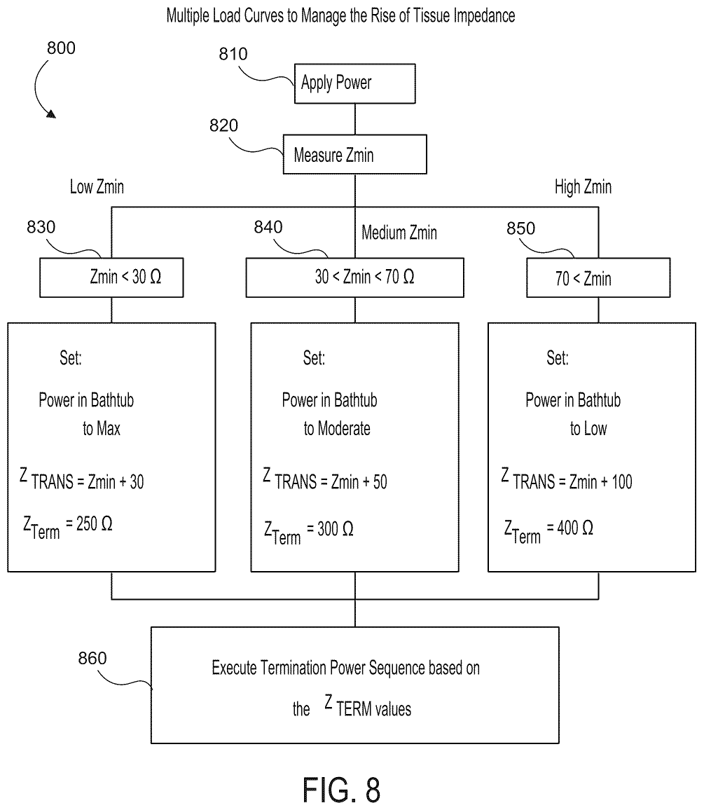

Referring to FIG. 8, in some aspects, the surgical instrument 110 may be configured to apply different power algorithms to manage the rise of tissue impedance, based on varying levels of initial tissue impedance at the surgical site. The flowchart 800 provides an example of how multiple load curves may be programmed into the surgical instrument 110 to apply different power adjustments based on impedance measurements during the sealing procedures. For example, an algorithm to adjust the power may begin at block 810 that includes applying power to the end effector and ultimately to the tissue in question at the surgical site. At block 820, while the end effector is applying the electrosurgical energy to the tissue, the impedance may be measured, and the minimum impedance may be determined based on the point at which the impedance eventually begins to rise. The end effector may include one or more sensors configured to monitor the impedance, voltage, or current, and may apply a number of mathematical formulas to determine what the measured impedance is. Various examples for how the tissue impedance may be measured are described in U.S. patent application Ser. Nos. 14/230,349 and 14/660,620, which are incorporated herein by reference.

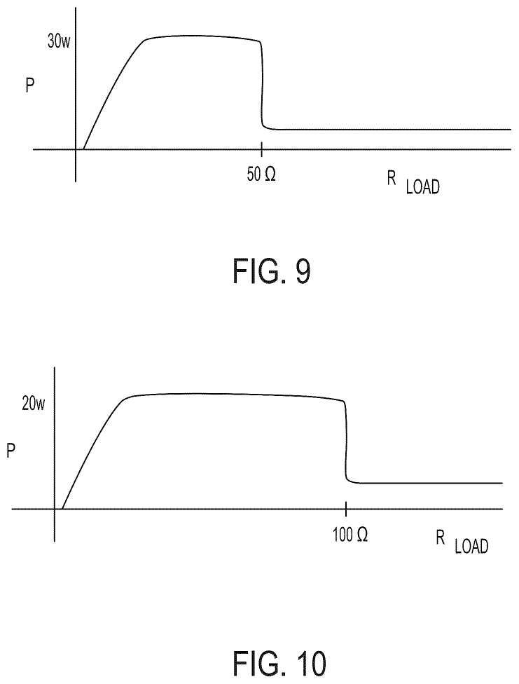

In this example, three different power profiles may be available to be applied to the tissue, based on the measured level of minimum impedance: low, medium, and high. In this example, at block 830, the low minimum impedance threshold is defined as when the minimum impedance is less than 30 ohms. At block 840, the medium minimum impedance threshold is defined as when the minimum impedance is between 30 and 70 ohms. At block 850, the high minimum impedance threshold is defined as when the minimum impedance is greater than 70 ohms. Based on the measured minimum impedance falling into one of these three categories, the load curve may be adjusted according to three characteristics. For example, if following the low minimum impedance profile, the power level in the bathtub region may be set to a maximum available power level (e.g., 30 W), the transition impedance threshold may be defined as 30 ohms greater than the measured minimum impedance, and the impedance at which all power is terminated may be set to 250 ohms. Based on these three characteristics, the power load curve may be generated. Examples of these different load curves are visually depicted in FIGS. 9 and 10, below. As another example, if following the medium minimum impedance profile, the power level in the bathtub may be set to a moderate available power level (e.g., 20 W), the transition impedance threshold may be defined as 50 ohms greater than the measured minimum impedance, and the impedance at which all power is terminated may be set to 300 ohms. As a third example, if following the high minimum impedance profile, the power level in the bathtub region may be set to a low available power level (e.g., 10 W), the transition impedance threshold may be defined as 100 ohms greater than the measured minimum impedance, and the impedance at which all power is terminated may be set to 400 ohms.

In some aspects, rather than the minimum impedance being measured, and initial impedance may be measured and the load curves may be varied based on measured initial impedance levels. It may be apparent to those with skill in the art how the examples provided herein may be modified to account for an initial impedance level being measured, rather than the minimum impedance level being measured. For example, the calculation of the transition impedance may be offset by a different factor to account for the difference in value between the minimum impedance and the initial impedance. In addition, the thresholds under which the different load curves may be followed (e.g., blocks 830, 840, and 850) may be based on modified criteria according to the different ranges of initial impedance.

The power system in the medical instrument 110 may apply power to the tissue according to the load curve, depending on which load curve is chosen. In all cases, the power system may be configured to taper the power and the bathtub region as the impedance begins to slowly rise toward the transition impedance level, consistent with the concepts described in FIGS. 6A and 7.

Once the medical instrument 110 has finished applying power according to one of the load curves, a termination procedure may be executed at block 860. In some aspects, the termination power sequence may be based on what termination impedance value was set in the previous blocks of flowchart 800. For example, a series of termination pulses may be transmitted to the end effector of the medical instrument 110.

Referring to FIG. 9, a visual depiction of an example load curve under the low minimum impedance threshold is shown (see FIG. 8), according to some aspects. In this example, it may have been determined that the minimum impedance is 20 ohms, and therefore that the transition impedance level is 50 ohms (i.e., 30 ohms greater than the minimum impedance). The power level in the bathtub region may be set to a maximum level, such as 30 W. After the transition impedance level is reached, the power level may be dropped to a minimum, and may be ultimately terminated once the impedance reaches 250 ohms (not shown). In some aspects, the power level is dramatically cut once the transition impedance level is reached, while in other cases the power level may be tapered to decrease more gradually prior to the transition impedance being reached.

Referring to FIG. 10, a visual depiction of an example load curve under the moderate impedance threshold is shown (see FIG. 8), according to some aspects. In this example, it may have been determined that the minimum impedance is 50 ohms, and therefore that the transition impedance level is 100 ohms (i.e., 50 ohms greater than the minimum impedance). The power level in the bathtub region may be set to a moderate level, such as 20 W. After the transition impedance level is reached, the power level may be dropped to a minimum, and may be ultimately terminated once the impedance reaches 300 ohms (not shown). In some aspects, the power level is dramatically cut once the transition impedance level is reached, while in other cases the power level may be tapered to decrease more gradually prior to the transition impedance being reached.

In general, the example power algorithms and concepts from which these examples are based on may be adapted to many different types of electrode configurations, and aspects are not so limited. Various examples include electrodes of various length and width, including wider, narrower, longer or shorter electrodes than the examples shown herein; electrodes using I-Beam technology; motorized electrosurgical systems (similar to those described herein); scissor-type electrodes; and hand-held forceps-like instruments, whether open, laparoscopic or robotic. The power algorithms described herein may be set and adapted to these different scenarios by adjusting the various parameters as shown and described herein.

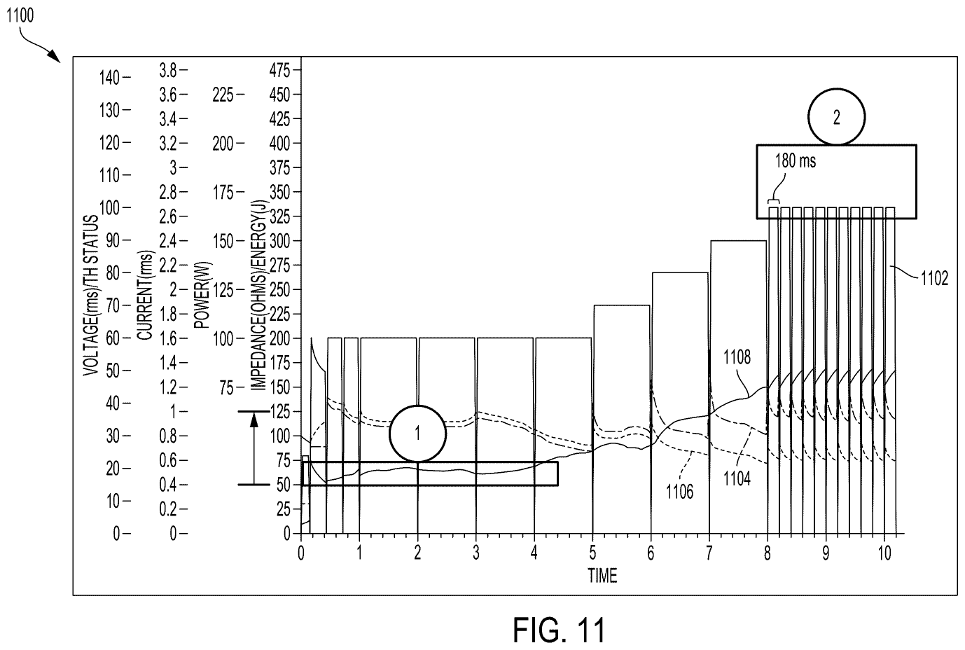

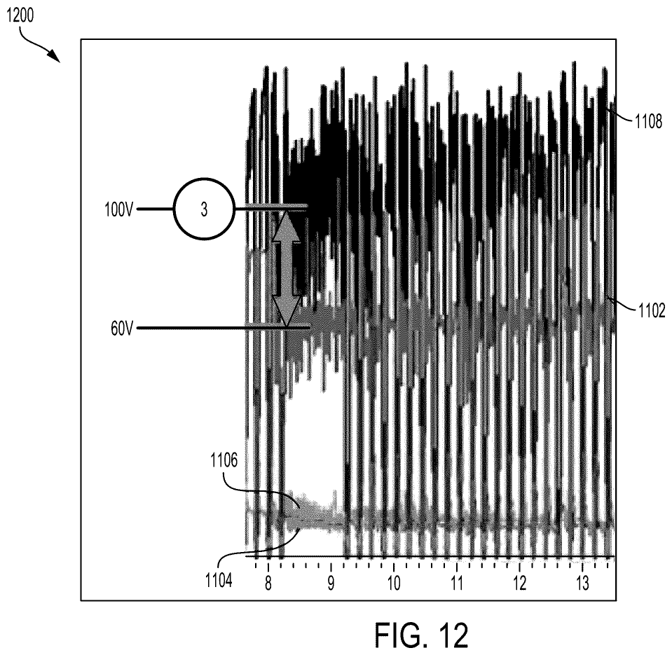

FIG. 11 is a graphical illustration 1100 of impedance threshold and minimum pulse duration showing an example of additional adjustments that can be made to varying the power to account for other tissue properties, according to one aspect of the present disclosure. The vertical axis represents, from left to right, Voltage (rms), Current (rms), Power (W), and Impedance (Ohms)/Energy (J) and the horizontal axis represents Time. Accordingly, a voltage curve 1102, current curve 1104, power curve 1106, and impedance/energy curve 1108 are shown superimposed as a function of time. Referring to FIG. 11, the illustration 1100 shows an example of additional adjustments that can be made to varying the power, in order to account for other tissue properties. The illustration 1100 shows example impedance thresholds and minimum pulse durations over time, along with corresponding levels of power, voltage and current. In some aspects, the initial power level applied to the tissue may be based on additional factors, and the power adjustments may be varied to prolong the bathtub region based on these initial varied power levels, according to some aspects. For example, fatty tissues tend to have higher impedances during sealing. These impedances are often greater than 50 ohms after the initial pulses of energy are delivered to the tissue. Without accounting for these tissue properties, if the tissue impedance is greater than 50 ohms, the tissue will not receive full power from the generator. In response, increasing this threshold from 50 to 125 ohms (see circle 1 in illustration 1100) may enable the generator to deliver full power into the base mesentery, thus reducing sealing cycle time. In this case, if human tissue is more resistive than 125 ohms, then long seal times could occur. The threshold may be therefore need to be increased beyond 125 ohms in other circumstances.

Regarding minimum pulse duration, it has been observed that with short 180 millisecond (ms) pulses, the impedance (black curve, e.g., curve within circle 1) tends to stall and not rise quickly (see the movement of this curve within the time span under circle 2). In order to allow the tissue impedance to increase, the duration of the pulse on time can be increased. Thus, in some aspects, the minimum pulse width may be increased from 180 ms to 480 ms for composite load curve (CLC) tables.

FIG. 12 is a graphical illustration 1200 of voltage cutback caused by an impedance threshold greater than 325 Ohms, according to one aspect of the present invention. As shown, the voltage curve 1102 based on the CLC table bounces between 60 and 100 Volts (see circle 3 in illustration 1100) as tissue impedance approaches a 300 Ohm termination impedance. This bouncing causes the impedance rise rate to slow down as can be seen in the bouncing impedance/energy curve 1108. The voltage cutback is caused by a 325 Ohm threshold programmed in the original algorithm. If the 325 Ohm threshold is exceeded, the generator is configured to reduce the applied voltage from 100 volts to 60 volts. Accordingly, one aspect of the algorithm described herein does not include an impedance threshold that is greater than 325 Ohms.

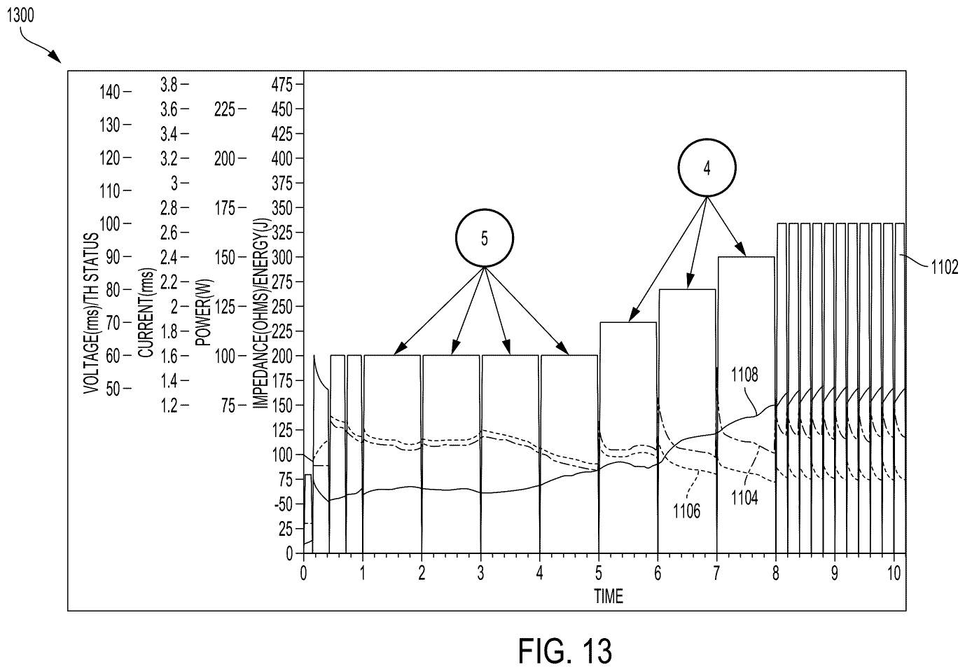

FIG. 13 is a graphical illustration 1300 of a power pulse region of the graphical illustration 1100 shown in FIG. 11, according to one aspect of the present disclosure. In one aspect, the algorithm is configured such that the duration of the power pulse is 1 second for each step of the voltage curve 1102. It was observed that during the later half of each 1 second pulse the rise rate (slope) of the impedance curve 1108 would decrease (see circle 4 in illustration 1300). Accordingly, in one aspect of the algorithm, the duration of the power pulse is decreased to 0.5 seconds for each step of the voltage curve 1102 to prevent the impedance trajectory from stalling during the power pulse. In accordance with the 0.5 seconds duration of the power pulse, the number of power pulses is reduced from 4 to 3 (see circle 5 in illustration 1300). The duration of each power pulse remains 1 second. This allows tissue which is done earlier to proceed through the algorithm and thus finish sooner.

In general, aspects of the present disclosure may allow for various types of adjustments to be made to the amount of electrosurgical energy applied to the tissue at the surgical site, based on measured levels of impedance in the surgical tissue. For example, the power algorithms made differ if the type of energy applied to the surgical tissue includes RF energy versus ultrasonic energy. The various characteristics of the load curves may need to be adjusted, e.g., what the maximum power level can be set to, what should the transition impedance be set to, when should the energy be terminated, at what level impedance should the power begin to taper off, etc., due to how the tissue may respond based on the different types of energy being applied to it. However, in general, the general shapes of the power profiles should remain consistent, and it may simply be a matter of determining what values should be set for the critical characteristics of the load curves, based on a measured minimum impedance or initial impedance, and in some aspects also based on various other characteristics of the types of energy applied to the tissue.

In some cases, various aspects may be implemented as an article of manufacture. The article of manufacture may include a computer readable storage medium arranged to store logic, instructions and/or data for performing various operations of one or more aspects. In various aspects, for example, the article of manufacture may comprise a magnetic disk, optical disk, flash memory or firmware containing computer program instructions suitable for execution by a general purpose processor or application specific processor. The aspects, however, are not limited in this context.

The functions of the various functional elements, logical blocks, modules, and circuits elements described in connection with the aspects disclosed herein may be implemented in the general context of computer executable instructions, such as software, control modules, logic, and/or logic modules executed by the processing unit. Generally, software, control modules, logic, and/or logic modules comprise any software element arranged to perform particular operations. Software, control modules, logic, and/or logic modules can comprise routines, programs, objects, components, data structures, and the like that perform particular tasks or implement particular abstract data types. An implementation of the software, control modules, logic, and/or logic modules and techniques may be stored on and/or transmitted across some form of computer-readable media. In this regard, computer-readable media can be any available medium or media useable to store information and accessible by a computing device. Some aspects also may be practiced in distributed computing environments where operations are performed by one or more remote processing devices that are linked through a communications network. In a distributed computing environment, software, control modules, logic, and/or logic modules may be located in both local and remote computer storage media including memory storage devices.

Additionally, it is to be appreciated that the aspects described herein illustrate example implementations, and that the functional elements, logical blocks, modules, and circuits elements may be implemented in various other ways which are consistent with the described aspects. Furthermore, the operations performed by such functional elements, logical blocks, modules, and circuits elements may be combined and/or separated for a given implementation and may be performed by a greater number or fewer number of components or modules. As will be apparent to those of skill in the art upon reading the present disclosure, each of the individual aspects described and illustrated herein has discrete components and features which may be readily separated from or combined with the features of any of the other several aspects without departing from the scope of the present disclosure. Any recited method can be carried out in the order of events recited or in any other order which is logically possible.

Unless specifically stated otherwise, it may be appreciated that terms such as “processing,” “computing,” “calculating,” “determining,” or the like, refer to the action and/or processes of a computer or computing system, or similar electronic computing device, such as a general purpose processor, a DSP, ASIC, FPGA, or other programmable logic device, discrete gate or transistor logic, discrete hardware components, or any combination thereof designed to perform the functions described herein that manipulates and/or transforms data represented as physical quantities (e.g., electronic) within registers and/or memories into other data similarly represented as physical quantities within the memories, registers, or other such information storage, transmission, or display devices.

It is worthy to note that some aspects may be described using the expression “coupled” and “connected” along with their derivatives. These terms are not intended as synonyms for each other. For example, some aspects may be described using the terms “connected” and/or “coupled” to indicate that two or more elements are in direct physical or electrical contact with each other. The term “coupled,” however, also may mean that two or more elements are not in direct contact with each other, but yet still co-operate or interact with each other. With respect to software elements, for example, the term “coupled” may refer to interfaces, message interfaces, and application program interface, exchanging messages, and so forth.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Although various aspects have been described herein, many modifications, variations, substitutions, changes, and equivalents to those aspects may be implemented and will occur to those skilled in the art. Also, where materials are disclosed for certain components, other materials may be used. It is therefore to be understood that the foregoing description and the appended claims are intended to cover all such modifications and variations as falling within the scope of the disclosed aspects. The following claims are intended to cover all such modification and variations.

1. A surgical system comprising:
an end effector comprising at least one energy delivery component configured to transmit electrosurgical energy to a tissue at a surgical site; and
a control circuit communicatively coupled to the at least one energy delivery component and programmed to:
for a first application period, cause the at least one energy delivery component to transmit the electrosurgical energy at a first power level, the first application period comprising a point in time where impedance of the tissue reaches a minimum impedance value; and
for a second application period after the first application period, cause the at least one energy delivery component to transmit the electrosurgical energy starting from a predetermined proportion of a transition impedance threshold level and decreasing the electrosurgical energy at a steady rate from the first power level until a second power level is reached, wherein the second power level is lower than the first power level and the second application period comprises a point in time where the impedance of the tissue rises above the minimum impedance value.

2. The surgical system of claim 1, wherein the first application period and the second application period combined comprise a time period where the electrosurgical energy is delivered at a higher rate and causes sealing of the tissue at the surgical site.

3. The surgical system of claim 21, wherein the third application period further comprises a time period where the impedance of the tissue rises to a level such that the electrosurgical energy is delivered at a lower rate and no longer causes sealing of the tissue at the surgical site.

4. The surgical system of claim 1, further comprising at least one sensor configured to measure an initial level of impedance in the tissue and the minimum impedance value in the tissue.

5. The surgical system of claim 21, wherein the control circuit is further programmed to determine a beginning of the third application period based on a measured initial level of impedance in the tissue.

6. The surgical system of claim 21, wherein the control circuit is further programmed to determine a beginning of the third application period based on a measured minimum level of impedance in the tissue.

7. The surgical system of claim 1, wherein the first application period and the second application period combined comprise a continuous time period where the impedance of the tissue impedance remains below an initial level of impedance in the tissue.

8. The surgical system of claim 1, wherein the at least one energy delivery component is configured to transmit RF and ultrasonic energy.

9. A method for transmitting electrosurgical energy to a tissue at a surgical site by a surgical system, the method comprising:
causing, by an energy delivery component of the surgical system, the electrosurgical energy to be applied to the tissue;
measuring, by at least one sensor of the surgical system, a benchmark level of impedance of the tissue;
determining, among a plurality of power load curve algorithms, a power load curve algorithm to be applied to the energy delivery component, based on the measured benchmark level of impedance of the tissue;
based on the determined power load curve algorithm:
for a first application period, causing the energy delivery component to transmit the electrosurgical energy at a first power level, the first application period comprising a point in time where impedance of the tissue reaches a minimum impedance value; and
for a second application period after the first application period, causing the energy delivery component to transmit the electrosurgical energy, starting from a predetermined proportion of a transition impedance threshold level and decreasing the electrosurgical energy at a steady rate from the first power level until a second power level is reached, wherein the second power level is lower than the first power level and the second application period comprises a point in time where the impedance of the tissue rises above the minimum impedance value.

10. The method of claim 9, wherein determining the power load curve algorithm comprises determining whether the benchmark level of impedance is less than a first threshold impedance value, whether the benchmark level of impedance is greater than the first threshold impedance value and less than a second threshold impedance value, and whether the benchmark level of impedance is greater than the second threshold impedance value.

11. The method of claim 9, wherein the first application period and the second application period combined comprise a time period where the electrosurgical energy is delivered at a higher rate and causes sealing of the tissue at the surgical site.

12. The method of claim 23, wherein the third application period further comprises a time period where the impedance of the tissue rises to a level such that the electrosurgical energy is delivered at a lower rate and no longer causes sealing of the tissue at the surgical site.

13. The method of claim 9, wherein the benchmark level of impedance is the minimum impedance value or an initial level of impedance of the tissue.

14. The method of claim 23, wherein a beginning of the third application period is based on the measured benchmark level of impedance.

15. The method of claim 9, wherein the first application period and the second application period combined comprise a continuous time period where the tissue impedance remains below an initial level of impedance in the tissue.

16. The method of claim 9, wherein the energy delivery component is configured to transmit RF and ultrasonic energy.

17. A surgical instrument comprising:
a handle assembly;
a shaft coupled to the handle assembly;
an end effector coupled to the shaft and comprising at least one energy delivery component configured to transmit electrosurgical energy to a tissue at a surgical site; and
a control circuit communicatively coupled to the energy delivery component and programmed to:
for a first application period, cause the at least one energy delivery component to transmit the electrosurgical energy at a first power level, the first application period comprising a point in time where impedance of the tissue reaches a minimum impedance value; and
for a second application period after the first application period, cause the at least one energy delivery component to transmit the electrosurgical energy, starting from a predetermined proportion of a transition impedance threshold level and decreasing the electrosurgical energy at a steady rate from the first power level until a second power level is reached, wherein the second power level is lower than the first power level and the second application period comprises a point in time where the impedance of the tissue rises above the minimum impedance value.

18. The surgical instrument of claim 17, wherein the first application period and the second application period combined comprise a time period where the electrosurgical energy is delivered at a higher rate and causes sealing of the tissue at the surgical site.

19. The surgical instrument of claim 25, wherein the third application period further comprises a time period where the impedance of the tissue rises to a level such that the electrosurgical energy is delivered at a lower rate and no longer causes sealing of the tissue at the surgical site.

20. The surgical instrument of claim 17, further comprising at least one sensor configured to measure an initial level of impedance in the tissue and the minimum impedance value in the tissue.

21. The surgical system of claim 1, wherein the control circuit is further programmed to:
for a third application period after the second application period, cause the at least one energy delivery component to transmit the electrosurgical energy at a third power level, wherein the third power level is lower than the second power level and the third application period comprises a point in time where the impedance of the tissue rises above the transition impedance threshold level.

22. The surgical system of claim 21, wherein the control circuit is further programmed to:
for the third application period after the second application period, directly after reaching the second power level, cause the at least one energy delivery component to reduce the second power level to the third power level while maintaining a constant level of the impedance of the tissue to transmit the electrosurgical energy at the third power level upon the impedance of the tissue reaching the transition impedance threshold level.

23. The method of claim 9, further comprising:
based on the determined power load curve algorithm:
for a third application period after the second application period, causing the energy delivery component to transmit the electrosurgical energy at a third power level, wherein the third power level is lower than the second power level and the third application period comprises a point in time where the impedance of the tissue rises above the transition impedance threshold level.

24. The method of claim 23, further comprising:
based on the determined power load curve algorithm:
for the third application period after the second application period, directly after reaching the second power level, causing the energy delivery component to reduce the second power level to the third power level while maintaining a constant level of the impedance of the tissue to transmit the electrosurgical energy at the third power level upon the impedance of the tissue reaching the transition impedance threshold level.

25. The surgical instrument of claim 17, wherein the control circuit is further programmed to:
for a third application period after the second application period, cause the at least one energy delivery component to transmit the electrosurgical energy at a third power level, wherein the third power level is lower than the second power level and the third application period comprises a point in time where the impedance of the tissue rises above the transition impedance threshold level.

26. The surgical instrument of claim 25, wherein the control circuit is further programmed to:
for the third application period after the second application period, directly after reaching the second power level, cause the at least one energy delivery component to reduce the second power level to the third power level while maintaining a constant level of the impedance of the tissue to transmit the electrosurgical energy at the third power level upon the impedance of the tissue reaching the transition impedance threshold level.
